(19) United States
(12) Patent Application Publication
(10) Pub. No.: US 2015/0118718 A1
(43) Pub. Date: Apr. 30, 2015

(54) STRAIN OF BACILLUS SUBTILIS AND APPLICATIONS THEREOF

(71) Applicant: NATIONAL CENTRAL UNIVERSITY, Taoyuan County (TW)

(72) Inventors: SHIR-LY HUANG, Taipei City (TW); SHENG-HSIN CHOU, Taoyuan County (TW)

(21) Appl. No.: 14/591,565

(22) Filed: Jan. 7, 2015

Related U.S. Application Data
(62) Division of application No. 13/905,460, filed on May 30, 2013, now abandoned.

(30) Foreign Application Priority Data
Nov. 30, 2012 (TW) 101145186

(51) Int. Cl. C12P 19/14; C12P 19/02

(52) U.S. Cl. CPC C12P 19/14; C12P 19/02
USPC 435/99

(57) ABSTRACT

The present invention is directed to a strain of Bacillus subtilis and applications using the strain to raise the efficiency and/or yield of generation of glucose produced by the hydrolysis of cellulose.

### CROSS-REFERENCE TO RELATED APPLICATION AND CLAIM OF PRIORITY

The application is a divisional of U.S. patent application Ser. No. 13/905,460, which was filed May 30, 2013 and claimed the benefit of Taiwan Patent Application No. 101145186, filed on Nov. 30, 2012 in the Taiwan Intellectual Property Office, both of which are incorporated herein by reference as if fully set forth.

### TECHNICAL FIELD

The present disclosure is directed to a novel strain of Bacillus subtilis SH44 and the applications thereof.

### BACKGROUND

Farmers in Taiwan often mix agricultural waste such as rice straw or paddy with stool of chicken or livestock to cause the waste to be fermented into compost. People accordingly notice that the microorganisms in the stool can decompose plant fiber, wherein microorganisms existing in the stools of herbivorous animals have the best efficiency of decomposition. In order to take into account environmental protection and development of alternative energy, people began to investigate biofuels for the development of alternative energy. Those biofuels mainly rely on the activities of various microorganisms for decomposing biomass, and some of those microorganisms exist in the digestive systems of herbivores, e.g. cattle, to be endosymbiosis for providing digestive function to the host.

So far, the researchers investigate intestinal commensal bacteria of human, pigs, mice, cockroaches, bison, beef cattle, sheep, goats, rabbits and zebras, etc., wherein ruminants of herbivores are most investigated. Herbivores have characteristic that the foods they intake are plants being rich in cellulose, hemicellulose and lignin, and those tough plant fibers are decomposed and transformed into essential nutrients to maintain the metabolism of their body. As the result of the evolution, the gastrointestinal tract of herbivores has specialized into a digestive system being suitable for decomposing plant fibers. Some herbivores can ruminate to re-digest the preliminarily digested plant fibers by the rumen, some others without rumen can even ferment the plant fibers in their ceca and rectums to obtain sufficient nutrients accordingly. The above-mentioned physiological characteristics are mainly dependent on various enzymes secreted by intestinal bacteria lived in the digestive system to decompose plant fibers to achieve the mutualism.

Many references screen the cellulolytic microorganisms from the cattle's rumen fluid and such experimental material is convenient to obtain by creating an opening on the rumen. However, there are seldom references relating to those herbivores without rumen such as camels, zebras and giraffes which do the fermentation by their ceca and rectums, since most of these animals are wild ones living in the natural environment. They are not easily to be the study object like raised cattle.

On the other hand, the lignocellulose is the biomass having the highest contents in the world and containing cellulose, hemicellulose and lignin. Via cellulase, the cellulose can also release pentoses therefrom which can be refined by microorganisms into the commercial product to replace those made of petrochemical raw materials.

Cellulase is usually generated by the microorganisms (e.g., fungi, bacteria and actinomycetes bacteria) and contains enzymes such as endoglucanase, exoglucanase and beta-glucosidase. The hydrolysis of cellulose is completed by these three enzymes jointly, i.e. (1) endoglucanase (EC 3.2.1.4): attacks non-crystalline regions of the cellulose so as to generate free short-chain polymerized sugar, (2) exoglucanase (EC 3.2.1.91): cuts the free short-chain polymerized sugar into cellobiose from the terminal thereof and (3) beta-glucosidase (EC 3.2.1.21): hydrolyze the cellobiose into glucose. When having respective proper contents, these three enzymes can jointly hydrolyze the cellulose into glucose, where the glucose can be further fermented into bioethanol. However, the biggest obstacle for the promotion of bioethanol is that these enzymes need high doses for the hydrolysis and the prices thereof are very expensive. Moreover, the endoglucanase usually has a less content in the cellulase produced by microorganisms. For example, the commercial cellulase produced by strain of Trichoderma reesei Rut C-30 contains 80% of exoglucanase in total cellulase and only contains about 10-20% of endoglucanase in total cellulase. Since the respective contents of endoglucanase and exoglucanase in the total cellulase of T. reesei Rut C-30 have great difference, the endoglucanase would plays a more important role in the hydrolysis using the cellulase produced by strain of T. reesei Rut C-30.

Employing experiments and researches full-heartily and persistently, the applicant finally conceived strain of Bacillus subtilis and applications thereof.

### SUMMARY

The present disclosure is directed to a novel strain of Bacillus subtilis SH44 and the applications thereof.

On another aspect, the present disclosure provides a method for performing a hydrolysis of a cellulosic biomass, comprising steps of: providing the cellulosic biomass; providing a cellulase; providing a strain of Bacillus subtilis SH44; and mixing the cellulosic biomass, the cellulase and the strain of Bacillus subtilis SH44 to perform the hydrolysis.

On another aspect, the present disclosure provides a raw material of a hydrolysis of cellulose, comprising the cellulose and a strain of Bacillus subtilis SH44.

On another aspect, the present disclosure provides a strain of Bacillus subtilis SH44.

On another aspect, the present disclosure provides a mutant of the strain of Bacillus subtilis SH44.

### BRIEF DESCRIPTION OF THE DRAWINGS

FIG. 1 is a schematic diagram showing the clear ring of the growth of B. subtilis SH44 on the medium. Containing 11 is the colony of the strain, 12 indicates the clear zone generated via the hydrolysis of CMC in the medium.

FIG. 2 which is a microscope picture showing the morphology of i B. subtilis SH44 grown on the medium and stained via Gram staining.

FIG. 3 is the sequence of 16S rRNA of B. subtilis SH44 (SEQ ID NO.: 1).

FIG. 4 shows the result of the respective growing situations of B. subtilis SH44 incubated in different broth media.

FIG. 5 shows the result of the respective growing situations of B. subtilis SH44 incubated in different initial pH values of the LB media.

FIG. 6 shows the result of the respective growing situations of B. subtilis SH44 incubated in different temperatures in LB medium.

FIG. 7A shows the changes of concentration of glucose from 0^{th} to 96^{th} hours with or without the addition of strain SH44 in hydrolysis of rice straw. FIG. 7B shows the concentration of glucose at 96^{th} hour in the respective hydrolytic solutions of with adding medium (+SH44) and without (−SH44) groups between which the difference is B. subtilis SH44 being added therein or not.

### DETAILED DESCRIPTION

The present disclosure can be fully understood and accomplish by the skilled person according to the following embodiments. However, the practice of the present method is not limited into following embodiments.

The “hydrolysis of cellulose” or “hydrolysis of cellulosic biomass” described in the present disclosure means the reaction of producing the glucose of which the celluloses are taken as the raw material and the cellulase is involved for the catalysis.

The “mutant” described in the present disclosure means the mutated strains of which the mutation(s) is (are) caused naturally or by genetic engineering.

The present disclosure is directed to a novel strain of Bacillus subtilis SH44 and the applications thereof. B. subtilis SH44 is an isolated strain screened and isolated from the stool of camel (Hsinchu, Taiwan) and having characteristics of white colony, appearance of short-rod and growth in broth under 25° C. to 70° C.

B. subtilis SH44 was deposited in Bioresource Collection and Research Center of Food Industry Research and Development Institute of Republic of China (R.O.C.) and had depositing number of BCRC 910566.

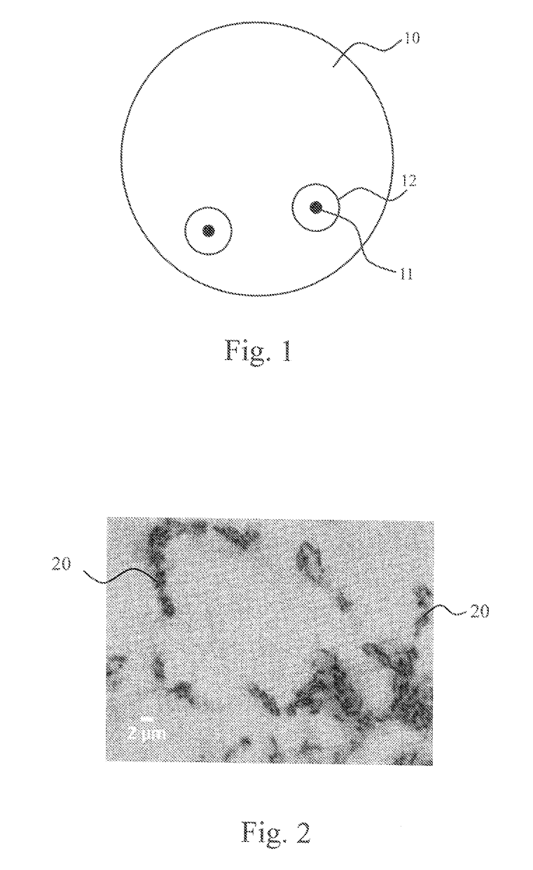

Please refer to FIG. 1 which a schematic diagram is showing the status of growth of B. subtilis SH44 on the medium. Specifically, 0.1 mg of the freeze-dried and homogenized stool of camel is added into sterile water and then vortex the mixture. After standing for 10 minutes, the mixture is separated into several parts which are further diluted into various concentrations from 10⁻⁸ to 10⁻¹⁰ respectively and then incubated on nutrient agars for estimating the colony forming unit (CFU) of the stool sample per gram. The colonies screened from the stool sample are seeded on solid nutrient agar containing celluloses and then the seeded microorganisms are incubated in the incubator at 50° C. After 24 hours, the colonies of microorganisms on the agar are gently scraped and then the agar is stained by Congo red solution 0.1% (w/w) by staining for 60 minutes. Subsequently, the Congo red solution on the agar is drained and the agar is then washed by NaCl water solution (1 M). If the microorganism has ability of decomposing the cellulose, the clear ring surrounding the location of colony of this microorganism and having a lighter color than that at other location stained by Congo red solution without colony can be observed. That is, the microorganism has ability of decomposing the cellulose can be screened and isolated by the above-mentioned procedures. Besides, the activity of decomposition of cellulose of a specific microorganism can be estimated by analyzing the ratio diameters of the corresponding colony and clear ring.

In FIG. 1, colony of B. subtilis SH44 11 grown on the agar medium 10 containing cellulose would have a clear ring 12 therearound after the above-mentioned staining procedures, which shows that B. subtilis SH44 has ability of decomposing the cellulose.

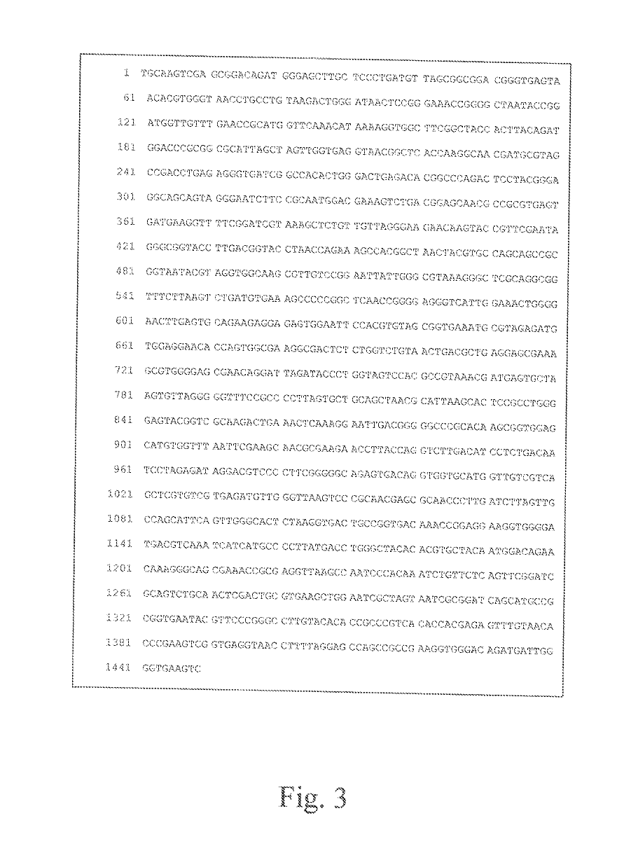

Please refer to FIG. 2 which is a picture taken by the microscope and showing the staining result of isolated B. subtilis SH44 grown on the medium and stained via Gram staining. As shown in FIG. 2, it is known that B. subtilis SH44 20 has an appearance of short-rod.

Please refer to FIG. 3 which shows the sequence, SEQ ID NO: 1, of 16S rRNA of B. subtilis SH44. Specifically, the 16S rRNA of B. subtilis SH44 is purified, amplified by PCR and then sequencing for obtaining the sequence thereof as shown in FIG. 3.

The sequence of 16S rRNA of B. subtilis SH44 is analyzed by sequence alignment via BLAST in GenBank (http://blast.ncbi.nlm.nih.gov/Btast.cgi) before the patent application. The result is shown in Table 1. Via the result of sequence alignment, it is known that the above-mentioned screened and isolated strain should be a Bacillus subtilis, and the strain number was designated SH44.

TABLE 1

| | | Max | Total | Query | E | Max |
|---|---|---|---|---|---|---|
| Accession | Description | score | score | coverage | value | ident |
| AP012496.1 | Bacillus subtilis BEST7003 DNA, complete genome | 2676 | 26630 | 100% | 0.0 | 100% |
| AP012495.1 | Bacillus subtilis BEST7613 DNA, complete genome | 2676 | 26636 | 100% | 0.0 | 100% |
| JX495609.1 | Bacillus sp. SNC1 16S ribosomal RNA gene, partial sequence | 2676 | 2676 | 100% | 0.0 | 100% |
| CP003783.1 | Bacillus subtilis QB928, complete genome | 2676 | 26730 | 100% | 0.0 | 100% |
| JX094283.1 | Bacillus subtilis strain AP254 16S ribosomal RNA gene, partial sequence | 2676 | 2676 | 100% | 0.0 | 100% |
| JN088185.1 | Bacillus sp. PPT 16S ribosomal RNA gene, partial sequence | 2676 | 2676 | 100% | 0.0 | 100% |
| JQ396173.2 | Bacillus subtilis subsp. subtilis strain KISR-1 16S ribosomal RNA gene, partial sequence | 2676 | 2676 | 100% | 0.0 | 100% |
| JQ308562.1 | Bacillus subtilis strain JPM18 16S ribosomal RNA gene, partial sequence | 2676 | 2676 | 100% | 0.0 | 100% |
| JQ435698.1 | Bacillus subtilis strain CE1 16S ribosomal RNA gene, partial sequence | 2676 | 2676 | 100% | 0.0 | 100% |
| GU972597.1 | Bacillus sp. LS03 16S ribosomal RNA gene, partial sequence | 2676 | 2676 | 100% | 0.0 | 100% |
| HE681728.1 | Bacillus subtilis partial 16S rRNA gene, isolate SG05 | 2676 | 2676 | 100% | 0.0 | 100% |
| JN366746.1 | Bacillus subtilis strain 30N2-5 16S ribosomal RNA gene, partial sequence | 2676 | 2676 | 100% | 0.0 | 100% |
| JQ081064.1 | Bacillus sp. EL31410 16S ribosomal RNA gene, partial sequence | 2676 | 2676 | 100% | 0.0 | 100% |
| AB513731.1 | Bacillus subtilis gene for 16S ribosomal RNA, partial sequence, strain: 318 | 2676 | 2676 | 100% | 0.0 | 100% |
| JN587510.1 | Bacillus subtilis strain K21 16S ribosomal RNA gene, partial sequence | 2676 | 2676 | 100% | 0.0 | 100% |
| GQ340505.1 | Bacillus sp. M40(2010) strain M40 16S ribosomal RNA gene, partial | 2676 | 2676 | 100% | 0.0 | 100% |
| | sequence >gb\|JN366711.1\| Bacillus subtilis strain 30L1-1 16S ribosomal RNA | | | | | |
| | gene, partial sequence >gb\|JN366754.1\| Bacillus subtilis subsp. subtilis | | | | | |
| | strain 30AA2-6 16S ribosomal RNA gene, partial sequence | | | | | |
| FR773878.1 | Bacillus subtilis partial 16S rRNAgene, strain CH1 | 2676 | 2676 | 100% | 0.0 | 100% |
| JF414762.1 | Bacillus subtilis strain BPRIST009 16S ribosomal RNA gene, partial sequence | 2676 | 2676 | 100% | 0.0 | 100% |
| JF414760.1 | Bacillus subtilis strain BPRIST007 16S ribosomal RNA gene, partial sequence | 2676 | 2676 | 100% | 0.0 | 100% |
| HQ851067.1 | Bacillus subtilis strain NBY44 16S ribosomal RNA gene, partial sequence | 2676 | 2676 | 100% | 0.0 | 100% |
| HM753632.1 | Bacillus subtilis subsp. subtilis strain WSR-KSU310 16S ribosomal RNA gene, partial | 2676 | 2676 | 100% | 0.0 | 100% |
| | sequence | | | | | |
| HM753628.1 | Bacillus subtilis subsp. subtilis strain WSE-KSU304 16S ribosomal RNA gene, partial | 2676 | 2676 | 100% | 0.0 | 100% |
| | sequence | | | | | |
| HQ423381.1 | Bacillus subtilis strain P6 16S ribosomal RNA gene, partial sequence | 2676 | 2676 | 100% | 0.0 | 100% |
| HQ423380.1 | Bacillus subtilis strain P4 16S ribosomal RNA gene, partial sequence | 2676 | 2676 | 100% | 0.0 | 100% |
| HQ236066.1 | Bacillus subtilis strain TAT1-8 16S ribosomal RNA gene, partial sequence | 2676 | 2676 | 100% | 0.0 | 100% |
| HM802140.1 | Bacillus subtilis strain SIO1 16S ribosomal RNA gene, partial sequence | 2676 | 2676 | 100% | 0.0 | 100% |
| HM214542.1 | Bacillus subtilis strain NB-01 16S ribosomal RNA gene, partial sequence | 2676 | 2676 | 100% | 0.0 | 100% |
| HM149534.1 | Bacillus subtilis strain WL-8 16S ribosomal RNA gene, partial sequence | 2676 | 2676 | 100% | 0.0 | 100% |
| GU216258.1 | Bacillus licheniformis strain KIBGE-IB1 16S ribosomal RNA gene, partial sequence | 2676 | 2676 | 100% | 0.0 | 100% |
| GU191916.1 | Bacillus subtilis subsp. subtilis strain SB 3130 16S ribosomal RNA gene, partial | 2676 | 2676 | 100% | 0.0 | 100% |
| | sequence | | | | | |
| GU191904.1 | Bacillus subtilis subsp. subtilis strain SB 3175 16S ribosomal RNA gene, partial | 2676 | 2676 | 100% | 0.0 | 100% |
| | sequence | | | | | |
| GU125628.1 | Bacillus subtilis strain IMAU80211 16S ribosomal RNA gene, partial sequence | 2676 | 2676 | 100% | 0.0 | 100% |
| GU125621.1 | Bacillus subtilis strain IMAU80203 16S ribosomal RNA gene, partial sequence | 2676 | 2676 | 100% | 0.0 | 100% |
| GQ871508.1 | Bacillus subtilis strain sdau08-96 16S ribosomal RNA gene, partial sequence | 2676 | 2676 | 100% | 0.0 | 100% |
| GQ421472.1 | Bacillus subtilis strain L4 16S ribosomal RNA gene, partial sequence | 2676 | 2676 | 100% | 0.0 | 100% |
| GQ402829.1 | Bacillus sp. G3(2009) 16S ribosomal RNA gene, partial sequence | 2676 | 2676 | 100% | 0.0 | 100% |
| GQ375227.1 | Bacillus subtilis subsp. subtilis strain CICC 10076 16S ribosomal RNA gene, partial | 2676 | 2676 | 100% | 0.0 | 100% |
| | sequence | | | | | |
| GQ199597.1 | Bacillus subtilis strain 1527 16S ribosomal RNA gene, partial sequence | 2676 | 2676 | 100% | 0.0 | 100% |
| AL009126.3 | Bacillus subtilis subsp. subtilis str. 168 complete genome | 2676 | 26621 | 100% | 0.0 | 100% |
| EU780682.1 | Bacillus subtilis strain WD23 16S ribosomal RNA gene, partial | 2676 | 2676 | 100% | 0.0 | 100% |
| | sequence >gb\|JN644487.1\| Bacillus subtilis strain BB14_2C 16S ribosomal RNA | | | | | |
| | gene, partial sequence | | | | | |
| AB440270.1 | Bacillus subtilis gene for 16S rRNA, partial sequence, strain: SR | 2676 | 2676 | 100% | 0.0 | 100% |
| AB440269.1 | Bacillus subtilis gene for 16S rRNA, partial sequence, strain: DB | 2676 | 2676 | 100% | 0.0 | 100% |
| AB440268.1 | Bacillus subtilis gene for 16S rRNA, partial sequence, strain: SB5 | 2676 | 2676 | 100% | 0.0 | 100% |
| AB440267.1 | Bacillus subtilis gene for 16S rRNA, partial sequence, strain: SB3 | 2676 | 2676 | 100% | 0.0 | 100% |
| AB440266.1 | Bacillus subtilis gene for 16S rRNA, partial sequence, strain: SB4 | 2676 | 2676 | 100% | 0.0 | 100% |
| EU660332.1 | Bacillus subtilis strain CM19 16S ribosomal RNA gene, partial sequence | 2676 | 2676 | 100% | 0.0 | 100% |
| EU660321.1 | Bacillus subtilis strain CM5 16S ribosomal RNA gene, partial sequence | 2676 | 2676 | 100% | 0.0 | 100% |
| EU684952.1 | Bacillus subtilis strain B215 16S ribosomal RNA gene, partial sequence | 2676 | 2676 | 100% | 0.0 | 100% |
| AB201120.1 | Bacillus subtilis gene for 16S rRNA, partial sequence, strain: LB-01 | 2676 | 2676 | 100% | 0.0 | 100% |
| EU221345.1 | Bacillus subtilis strain PAB1C8 16S ribosomal RNA gene, partial sequence | 2676 | 2676 | 100% | 0.0 | 100% |
| EU221334.1 | Bacillus subtilis strain JM1C6 16S ribosomal RNA gene, partial sequence | 2676 | 2676 | 100% | 0.0 | 100% |
| EU221333.1 | Bacillus subtilis strain JM1C5 16S ribosomal RNA gene, partial sequence | 2676 | 2676 | 100% | 0.0 | 100% |
| EU221332.1 | Bacillus subtilis strain JM1C1 16S ribosomal RNA gene, partial sequence | 2676 | 2676 | 100% | 0.0 | 100% |
| EU081774.1 | Bacillus subtilis strain PY79 tRNA-Arg gene, partial sequence; tRNA-Pro and tRNA- | 2676 | 2676 | 100% | 0.0 | 100% |
| | Ala genes, complete sequence; and recombinant ribosomal RNA operon, complete | | | | | |
| | sequence | | | | | |
| DQ993674.1 | Bacillus subtilis strain BCRC 10058 16S ribosomal RNA gene, partial sequence | 2676 | 2676 | 100% | 0.0 | 100% |
| AM237342.1 | Bacillus subtilis subsp. subtilis partial 16S rRNA gene, isolate OS-6.2 | 2676 | 2676 | 100% | 0.0 | 100% |
| DQ401073.1 | Bacillus subtilis strain Setapak 8 16S ribosomal RNA gene, partial sequence | 2676 | 2676 | 100% | 0.0 | 100% |
| DQ198162.1 | Bacillus subtilis WL-6 16S ribosomal RNA gene, partial sequence | 2676 | 2676 | 100% | 0.0 | 100% |
| DQ400916.1 | Bacillus subtilis strain 3A25 16S ribosomal RNA gene, partial sequence | 2676 | 2676 | 100% | 0.0 | 100% |
| AB188212.1 | Bacillus sp. TUT1206 gene for 16S rRNA, partial sequence | 2676 | 2676 | 100% | 0.0 | 100% |
| AY030331.1 | Bacillus subtilis strain KL-077 16S ribosomal RNA gene, partial | 2676 | 2676 | 100% | 0.0 | 100% |
| | sequence >dbj\|AB679315.1\| Bacillus subtilis gene for 16S rRNA, partial sequence, | | | | | |
| | strain: KUH-71 | | | | | |
| AY030330.1 | Bacillus subtilis strain KL-073 16S ribosomal RNA gene, partial sequence | 2676 | 2676 | 100% | 0.0 | 100% |
| DQ376027.1 | Bacillus subtilis 3xWMARB-4 16S ribosomal RNA gene, partial sequence | 2676 | 2676 | 100% | 0.0 | 100% |
| AB210982.1 | Bacillus subtilis gene for 16S rRNA, partial sequence, strain: SSCT51 | 2676 | 2676 | 100% | 0.0 | 100% |
| AB110598.1 | Bacillus subtilis gene for 16S rRNA, partial sequence | 2676 | 2676 | 100% | 0.0 | 100% |
| AF500205.1 | Bacillus sp. CJ11043 16S ribosomal RNA gene, partial sequence | 2676 | 2676 | 100% | 0.0 | 100% |
| AY995572.1 | Bacillus subtilis strain IDCC1105 16S ribosomal RNA gene, partial sequence | 2676 | 2676 | 100% | 0.0 | 100% |
| AY995568.1 | Bacillus subtilis strain IDCC 1101 16S ribosomal RNA gene, partial | 2676 | 2676 | 100% | 0.0 | 100% |
| | sequence >gb\|HM587993.1\| Bacillus subtilis strain BEC-1 16S ribosomal RNA | | | | | |
| | gene, partial sequence | | | | | |
| AY971364.1 | Bacillus subtilis strain CICC10147 16S ribosomal RNA gene, partial sequence | 2676 | 2676 | 100% | 0.0 | 100% |
| AY917141.1 | Bacillus subtilis strain CICC10076 16S ribosomal RNA gene, partial sequence | 2676 | 2676 | 100% | 0.0 | 100% |
| AY881645.1 | Bacillus subtilis strain CICC10073 16S ribosomal RNA gene, partial sequence | 2676 | 2676 | 100% | 0.0 | 100% |
| AY881638.1 | Bacillus subtilis strain CICC10028 16S ribosomal RNA gene, partial | 2676 | 2676 | 100% | 0.0 | 100% |
| | sequence >gb\|JN609214.1\| Bacillus subtilis strain MTCC WP34 16S ribosomal RNA | | | | | |
| | gene, partial sequence | | | | | |
| AB055853.1 | Bacillus sp. CH10-1 gene for 16S rRNA, partial sequence | 2676 | 2676 | 100% | 0.0 | 100% |
| AB055852.1 | Bacillus sp. CH7-1 gene for 16S rRNA, partial sequence | 2676 | 2676 | 100% | 0.0 | 100% |
| AB055851.1 | Bacillus sp. CH20-1 gene for 16S rRNA, partial sequence | 2676 | 2676 | 100% | 0.0 | 100% |
| AB055850.1 | Bacillus sp. CH19-3 gene for 16S rRNA, partial sequence | 2676 | 2676 | 100% | 0.0 | 100% |
| AB055849.1 | Bacillus sp. CH15-2 gene for 16S rRNA, partial sequence | 2676 | 2676 | 100% | 0.0 | 100% |
| AB055848.1 | Bacillus sp. CH4-5 gene for 16S rRNA, partial sequence | 2676 | 2676 | 100% | 0.0 | 100% |
| AB055846.1 | Bacillus sp. CH4-4 gene for 16S rRNA, partial sequence >dbj\|AB733579.1\| Bacillus sp. | 2676 | 2676 | 100% | 0.0 | 100% |
| | MBEU16 gene for 16S rRNA, partial sequence | | | | | |
| NR_027552.1 | Bacillus subtilis subsp. subtilis strain DSM 10 16S ribosomal RNA, partial | 2676 | 2676 | 100% | 0.0 | 100% |
| | sequence >emb\|AJ276351.1\| Bacillus subtilis 16S rRNA gene, | | | | | |
| | strain DSM10 | | | | | |
| D26185.1 | Bacillus subtilis gene, 180 kilobase region of replication origin | 2676 | 10655 | 100% | 0.0 | 100% |
| HM165188.1 | Bacillus sp. PS4 16S ribosomal RNA gene, complete sequence | 2675 | 2675 | 99% | 0.0 | 100% |
| FJ169948.1 | Bacillus sp. 7DU3 16S ribosomal RNA gene, partial sequence | 2675 | 2675 | 99% | 0.0 | 100% |
| AY881637.1 | Bacillus subtilis strain CICC10027 16S ribosomal RNA gene, partial sequence | 2675 | 2675 | 99% | 0.0 | 100% |
| JQ361055.1 | Bacillus subtilis strain CYBS-6 16S ribosomal RNA gene, partial sequence | 2673 | 2673 | 100% | 0.0 | 99% |
| GQ340479.1 | Bacillus amyloliquefaciens strain M16 16S ribosomal RNA gene, partial sequence | 2673 | 2673 | 100% | 0.0 | 99% |
| AB425345.1 | Bacillus sp. M307 gene for 16S rRNA, partial sequence | 2673 | 2673 | 99% | 0.0 | 100% |
| AB425344.1 | Bacillus sp. M306 gene for 16S rRNA, partial sequence | 2673 | 2673 | 99% | 0.0 | 100% |
| AB300816.1 | Bacillus subtilis gene for 16S rRNA, partial sequence, strain: Y7-1 | 2673 | 2673 | 99% | 0.0 | 100% |
| AB300813.1 | Bacillus subtilis gene for 16S rRNA, partial sequence, strain: W20 | 2673 | 2673 | 99% | 0.0 | 100% |
| AB325586.1 | Bacillus subtilis subsp. subtilis gene for 16S rRNA, partial sequence, strain: NBRC | 2673 | 2673 | 99% | 0.0 | 100% |
| | 101245 | | | | | |
| AM237355.1 | Bacillus subtilis subs. subtilis partial 16S rRNA gene, isolate OS-44.a | 2673 | 2673 | 100% | 0.0 | 99% |
| AB210989.1 | Bacillus subtilis gene for 16S rRNA, partial sequence, strain: SSCS2 | 2673 | 2673 | 100% | 0.0 | 99% |
| AB065370.1 | Bacillus subtilis gene for 16S rRNA, complete sequence | 2673 | 2673 | 100% | 0.0 | 99% |
| AB680179.1 | Bacillus subtilis gene for 16S rRNA, partial sequence, strain: NBRC 3936 | 2669 | 2669 | 99% | 0.0 | 99% |
| AB682183.1 | Bacillus subtilis gene for 16S rRNA, partial sequence, strain: NBRC 104443 | 2669 | 2669 | 99% | 0.0 | 99% |
| AB680377.1 | Bacillus subtilis gene for 16S rRNA, partial sequence, strain: NBRC | 2669 | 2669 | 99% | 0.0 | 99% |
| | 13169 >dbj\|AB680931.1\| Bacillus licheniformis gene for 16S rRNA, partial sequence, | | | | | |
| | strain: NBRC 15647 >dbj\|AB679982.1\| Bacillus subtilis gene for 16S rRNA, partial | | | | | |
| | sequence, strain: NBRC 3009 >dbj\|AB679983.1\| Bacillus subtilis gene for 16S rRNA, | | | | | |
| | partial sequence, strain: NBRC 3013 >dbj\|AB680029.1\| Bacillus subtilis gene for 16S | | | | | |
| | rRNA, partial sequence, strain: NBRC 3215 >dbj\|AB680066.1\| Bacillus subtilis gene for | | | | | |
| | 16S rRNA, partial sequence, strain: NBRC 3335 >dbj\|AB680067.1\| Bacillus subtilis gene | | | | | |
| | for 16S rRNA, partial sequence, strain: NBRC 3336 >dbj\|AB681480.1\| Bacillus subtilis | | | | | |
| | subsp. subtilis gene for 16S rRNA, partial sequence, strain: NBRC | | | | | |
| | 101581 >dbjAB681481.1\| Bacillus subtilis subsp. subtilis gene for 16S rRNA, partial | | | | | |
| | sequence, strain: NBRC 101582 >dbj\|AB681491.1\| Bacillus subtilis subsp. subtilis | | | | | |
| | gene for 16S rRNA, partial sequence, strain: NBRC 101592 | | | | | |
| HQ670762.1 | Bacillus subtilis strain Amp1 16S ribosomal RNA gene, partial sequence | 2669 | 2669 | 99% | 0.0 | 99% |
| AB374321.1 | Bacillus sp. TT401 gene for 16S rRNA, partial sequence | 2669 | 2669 | 99% | 0.0 | 99% |
| AB363740.1 | Bacillus subtilis gene for 16S rRNA, partial sequence, strain: NBRC 16449 | 2669 | 2669 | 99% | 0.0 | 99% |

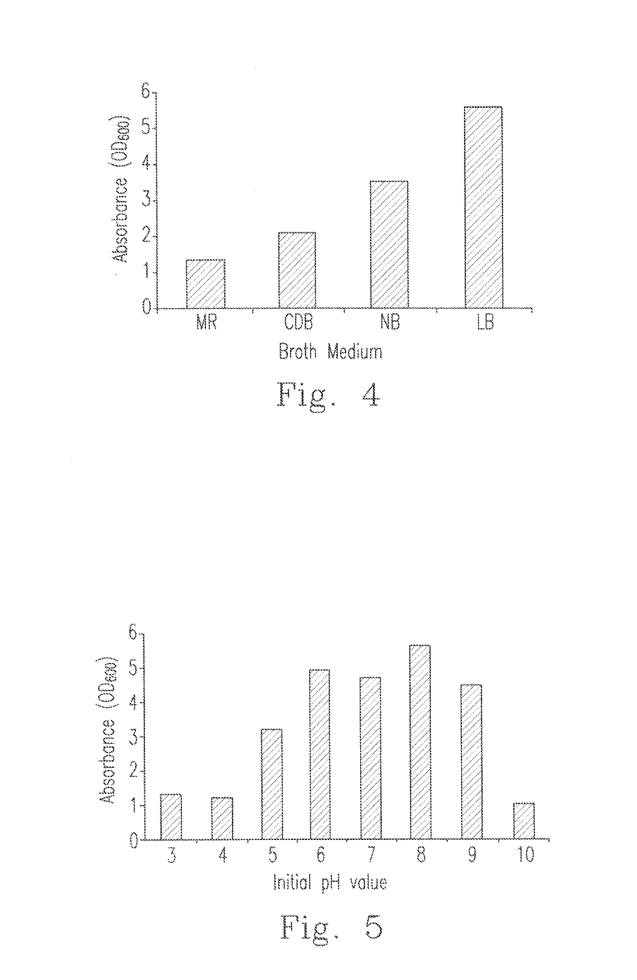

FIG. 4 shows an embodiment regarding the respective growing situations of B. subtilis SH44 incubated in different broth mediums. Under aseptic environment, the same amount of B. subtilis SH44 is seeded into mediums of Luria-Bertani (LB) medium, Nutrient broth (NB), the Czapek Dox broth (CDB) and Mandels-Reese medium (MR) (medium amount:seeding amount=10:1) and then those seeded strains are incubated at 37° C. At 8^{th} hour after seeding, the growth of B. subtilis SH44 incubated in those media are observed and measured via the turbidity of the medium which is represented by the absorbance at optical density (O.D.) 600 nm of the medium. In this embodiment, B. subtilis SH44 has a best growth in LB medium.

FIG. 5 shows an embodiment regarding the respective growing situations of B. subtilis SH44 incubated in different initial pH values of the broth medium. The respective initial pH values of LB mediums are adjusted to 3, 4, 5, 6, 7, 8, 9 and 10 by H₂SO₄ (1 N) or NaOH (1 N) solutions, and then those adjusted LB media are autoclaved and seeded with the same amount of B. subtilis SH44 under aseptic environment (medium amount:seeding amount=10:1). Those seeded strains of B. subtilis SH44 are incubated at 37° C. At 8^{th} hour after seeding, the growth of B. subtilis SH44 incubated in those mediums having different initial pH values are observed and measured via the turbidity of the medium which is represented by the absorbance at O.D. 600 nm of the medium. As shown in FIG. 5, B. subtilis SH44 can grow in a wild range of pH values. Also, it is known that the better range of pH value for the growth of B. subtilis SH44 is 5-9.

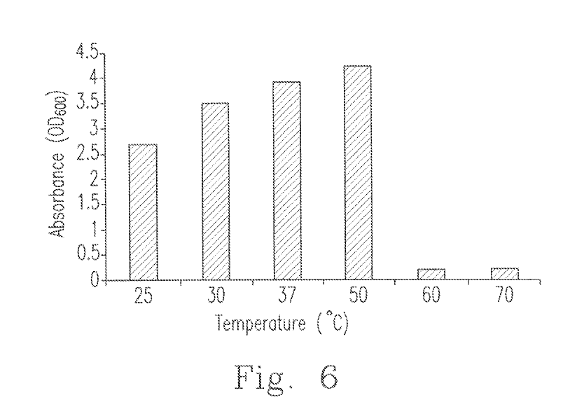

FIG. 6 shows an embodiment regarding the respective growing situations of B. subtilis SH44 incubated in different temperatures. Under aseptic environment, the same amount of B. subtilis SH44 is seeded into LB medium and then those seeded strains of B. subtilis SH44 are incubated at 25° C., 30° C., 37° C., 50° C., 60° C. and 70° C. At 8^{th} hour after seeding, the growth of B. subtilis SH44 incubated at different temperatures are observed and measured via the turbidity of the culture which is represented by the absorbance at O.D. 600 nm of the medium. As shown in FIG. 6, B. subtilis SH44 can grow in a wild range of temperature. From 25° C. to 55° C., the growth of B. subtilis SH44 is faster with the increase of the temperature. In this embodiment, B. subtilis SH44 appears a best growth at 50° C. Besides, B. subtilis SH44 can still grow slower at 60° C. and 70° C., which reveals that the B. subtilis SH44 can be applied to some processes which need to be performed under the environment with high temperature.

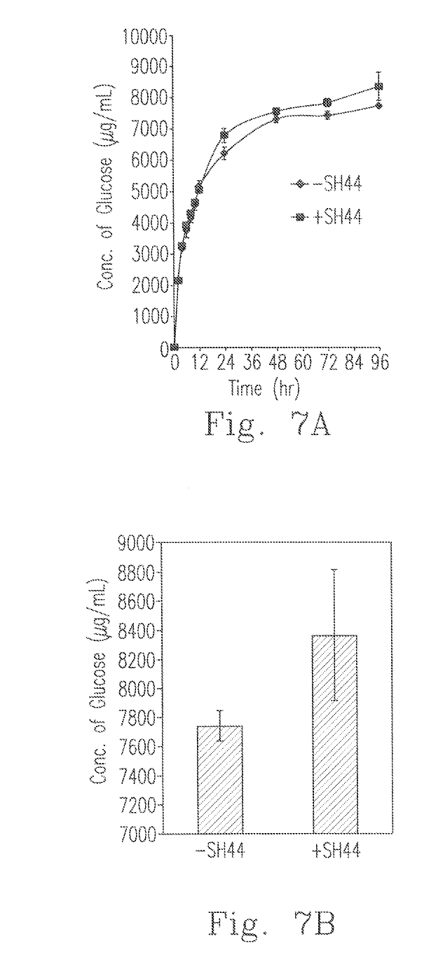

FIG. 7A shows the influence of decomposition of cellulose to generate the glucose by adding the B. subtilis SH44 or not. The rice straws pre-treated by dilute acid are mixed with the cellulase produced by Trichoderma species induced by dilute acid-pretreated lignocellulose, wherein the amount of cellulase is 15 FPU per gram of cellulose. Then, the mixture is put in the acetic acid buffer (0.05 M) containing carboxymethyl cellulose (CMC, 1 w/w %) to form a reactive mixture and then equally divided into two groups. One of the groups, group of +SH44, is added therein the LB broth in which the strains of B. subtilis SH44 have been incubated overnight (wherein the adding amount of B. subtilis SH44 is 46 mg of dry strain per 50 mL of reactive mixture). Another group, group of −SH44, is without adding any B. subtilis SH44.

The rice straws of these two groups are taken as the material for the enzymatic hydrolysis of cellulose. The hydrolysis is performed at 50° C. and the hydrolytic solution is taken at specific times. The taken hydrolytic solutions are analyzed by HPLC for measuring the glucose generated from the hydrolysis of cellulose and recorded as in FIG. 7A. In the group of +SH44, B. subtilis SH44 contained therein would grow and metabolize in the reactive mixture during the hydrolysis, which assists in the processing of the hydrolysis of cellulose.

As shown in FIG. 7A, the contents of glucose in the hydrolytic solution of group of +SH44 indeed starts to be higher than that of group of −SH44 from 12^{th} hour from the beginning of the hydrolysis. It appears that the addition of B. subtilis SH44 into the hydrolytic reaction can raise the efficiency of generation of glucose caused by the hydrolysis of cellulose.

Besides, as shown in FIG. 7B, at the 96^{th} hour from the beginning of the hydrolysis, the concentration of total glucose in the hydrolytic solution of −SH44 group is averaged at 7740 μg/mL which is obviously lower than that, 8632 μg/mL, of +SH44 group and for 8.04%. Evidently, the addition of B. subtilis SH44 into the hydrolytic reaction can raise the yield of product, i.e. glucose, of the hydrolysis of cellulose.

In the above-mentioned embodiments, the cellulosic biomass, rice straws, is applied to be the raw material for the hydrolysis of cellulose. In fact, the hydrolysis of any kind of material containing cellulose can be involved therein B. subtilis SH44 for raising the generating efficiency and/or yield of glucose during the hydrolytic reaction. For example, the cellulosic biomasses can be, but not limited, straw, rice chaff, straw, wheat bran, bagasse, pennisetum or timber.

Moreover, besides the pretreatment of biomass by diluted acid, other pretreatment method such as, steam explosion, alkali treatment, hydrothermal and others can be used separately or jointed in the present hydrolysis of cellulose.

The cellulose used in the present embodiments contains endoglucanase, exoglucanase and beta-glucosidase mixed with arbitrary radios and at least contains exoglucanase and beta-glucosidase.

Also, the mutant of B. subtilis SH44 retaining the abilities/characteristics of raising the generating efficiency and/or yield of glucose of the hydrolysis of cellulose is obviously included in the present invention. The seeding amount of the strain of B. subtilis SH44 or the mutant thereof is 5-10% (v/v) of the total reactive volume of hydrolysis or at least 1 mg of dry strain per 50 mL of the total reactive volume of hydrolysis.

Based on the above, it is apparent that the B. subtilis SH44 can raise the generating efficiency and/or yield of glucose of the hydrolysis of cellulose. Besides, since the B. subtilis SH44 can be used to assist in the hydrolysis of the cellulose, it can also be applied to the cut of cellulose. Accordingly, B. subtilis SH44 is worthy of the manufacture of special chemical or drug where the raw material of the special chemical or the drug contains the cellulose and the cellulose must be cut so as to produce the special chemical or the drug. Furthermore, the B. subtilis SH44 is applicable to the manufactures of glucose, biofuel, cellulosic ethanol, agricultural compost, animal feed and pulp, etc.

### Embodiments

Embodiment 1: A method for performing a hydrolysis of a cellulosic biomass, comprising steps of: providing the cellulosic biomass; providing a cellulase; providing a strain of Bacillus subtilis SH44; and mixing the cellulosic biomass, the cellulase and the strain of Bacillus subtilis SH44 to perform the hydrolysis.

Embodiment 2 is a method as described in Embodiment 1, wherein the hydrolysis is performed at a reaction temperature ranged from 25° C. to 70° C.

Embodiment 3 is a method as described in Embodiment 1, wherein the cellulosic biomass is pre-treated, e.g. with a dilute acid, before being reacted in the hydrolysis.

Embodiment 4 is a method as described in Embodiment 1, wherein the cellulase includes an exoglucanase and a beta-glucosidase.

Embodiment 5 is a method as described in Embodiment 4, wherein the cellulase further includes an endoglucanase.

Embodiment 6 is a method as described in Embodiment 1, wherein the hydrolysis is an enzymatic hydrolysis and generates a glucose.

Embodiment 7 is a method as described in Embodiment 1, wherein the hydrolysis has a product, the product is a glucose and the method is used to increase at least one of a yield and a generating efficiency of the glucose.

Embodiment 8: A method for cutting a cellulose, comprising steps of: providing the cellulose; providing a cellulase; providing a strain of Bacillus subtilis SH44; and mixing the cellulose, the cellulase and the strain of Bacillus subtilis SH44 to cut the cellulose.

Embodiment 9 is a method as described in Embodiment 8, wherein the cellulase includes an exoglucanase and a beta-glucosidase and the cellulose is contained in a raw material of a special chemical or a drug.

Embodiment 10: A raw material of a hydrolysis of a cellulose, comprising the cellulose and a strain of Bacillus subtilis SH44.

Embodiment 11 is a material as described in Embodiment 10 further comprising a cellulase.

Embodiment 12 is a material as described in Embodiment 11, wherein the cellulase includes an exoglucanase and a beta-glucosidase.

Embodiment 13 is a material as described in Embodiment 12, wherein the cellulase further includes an endoglucanase.

Embodiment 14: A strain of Bacillus subtilis SH44.

Embodiment 15 is a mutant of the strain of Bacillus subtilis SH44 as described in Embodiment 14.

Embodiment 16 is a mutant as described in Embodiment 15 having a characteristic of increasing at least one of a yield of a product and a reactive efficiency of a cellulosic hydrolysis.

Embodiment 17 is a mutant as described in Embodiment 16, wherein the cellulosic hydrolysis is an enzymatic hydrolysis.

Embodiment 18 is a mutant as described in Embodiment 16, wherein the cellulosic hydrolysis has materials of the mutant, a cellulosic biomass and a cellulase.

Embodiment 19 is a mutant as described in Embodiment 16, wherein the cellulase includes an exoglucanase and a beta-glucosidase.

Embodiment 20 is a mutant as described in Embodiment 16, wherein the cellulosic hydrolysis has materials of the mutant, a cellulose and a cellulase.

While the disclosure has been described in terms of what is presently considered to be the most practical and preferred embodiments, it is to be understood that the disclosure needs not be limited to the disclosed embodiments. Therefore, it is intended to cover various modifications and similar arrangements included within the spirit and scope of the appended claims, which are to be accorded with the broadest interpretation so as to encompass all such modifications and similar structures.

What is claimed is:

1. A method for performing a hydrolysis of a cellulosic biomass, comprising steps of:
providing the cellulosic biomass;
providing a cellulase;
providing a strain of Bacillus subtilis SH44; and
mixing the cellulosic biomass, the cellulase and the strain of Bacillus subtilis SH44 to perform the hydrolysis.

2. The method as claimed in claim 1, wherein the hydrolysis is performed at a reaction temperature ranged from 25° C. to 70° C.

3. The method as claimed in claim 1, wherein the cellulosic biomass is pre-treated before being reacted in the hydrolysis.

4. The method as claimed in claim 1, wherein the cellulase includes an exoglucanase and a beta-glucosidase.

5. The method as claimed in claim 4, wherein the cellulase further includes an endoglucanase.

6. The method as claimed in claim 1, wherein the hydrolysis is an enzymatic hydrolysis and generates a glucose.

7. The method as claimed in claim 1, wherein the hydrolysis has a product, the product is a glucose and the method is used to increase at least one of a yield and a generating efficiency of the glucose.

8. A method for cutting a cellulose, comprising steps of:
providing the cellulose;
providing a cellulase;
providing a strain of Bacillus subtilis SH44; and
mixing the cellulose, the cellulase and the strain of Bacillus subtilis SH44 to cut the cellulose.

9. The method as claimed in claim 8, wherein the cellulase includes an exoglucanase and a beta-glucosidase and the cellulose is contained in a raw material of a special chemical or a drug.

10. A method for preparing a raw composition for producing a glucose by a hydrolysis, comprising steps of:
providing a cellulose;
providing a biologically pure culture of strain Bacillus subtilis BCRC 910566;
providing a cellulase consisting of an exoglucanase and a beta-glucosidase, wherein neither the exoglucanase nor the beta-glucosidase is produced by the biologically pure culture of strain Bacillus subtilis BCRC 910566; and
mixing the cellulose, the biologically pure culture of strain Bacillus subtilis BCRC 910566, the exoglucanase and the beta-glucosidase.


SEQUENCE LISTING (1 sequence)

SEQ ID NO: 1 - DNA, 1449 bp, Bacillus subtilis
tgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtc